(19) United States
(12) Patent Application Publication
(10) Pub. No.: US 2022/0133223 A1
(43) Pub. Date: May 5, 2022

(54) SUMMARIAL SCORES FOR AN EMR PLATFORM

(71) Applicant: Children's Medical Center Corporation, Boston, MA (US)

(72) Inventors: John Nagi Kheir, Charlestown, MA (US); Anjuli Melissa Sinha, Philadelphia, PA (US); Sarah-Jane van den Bosch, Newton, MA (US)

(73) Assignee: Children's Medical Center Corporation, Boston, MA (US)

(21) Appl. No.: 17/431,371

(22) PCT Filed: Feb. 13, 2020
(86) PCT No.: PCT/US2020/018071

§ 371 Date: Aug. 16, 2021

Related U.S. Application Data
(60) Provisional application No. 62/806,540, filed on Feb. 15, 2019.

(51) Int. Cl. A61B 5/00; A61B 5/08; A61B 5/0205; G16H 50/30; G16H 20/10; G16H 10/60; G16H 40/20

(52) U.S. Cl. CPC A61B 5/4839; A61B 5/0816; A61B 5/0205; G16H 40/20; G16H 50/30; G16H 20/10; G16H 10/60; A61B 5/743

(57) ABSTRACT

Among other things, a respiratory support score is determined. Operating parameters of one or more respiratory support devices associated with a patient are received. Based on the operating parameters, a respiratory support type associated with the patent is determined. A score range for the respiratory support type is received, and a respiratory support score within the range is determined based on at least a subset of the operating parameters. In another aspect, a medication efficacy score is determined. A time point corresponding to a clinical action performed on a patient is identified, the clinical action including administration of a drug at a particular dosage. Parameters indicative of a clinical condition of the patient are tracked for a time range that includes the time point corresponding to the clinical action. Based on the parameters, a metric indicative of an efficacy of the clinical action performed on the patient is determined.

### CROSS-REFERENCE TO RELATED APPLICATION

This application claims priority to U.S. Provisional Patent Application No. 62/806,540, filed Feb. 15, 2019, the entire content of which is incorporated herein by reference.

### TECHNICAL FIELD

This description generally relates to an electronic medical record platform that can determine the degree of respiratory support that a patient requires at a certain point in time and a medication efficacy that describes how effective a medication was in achieving an intended endpoint.

### BACKGROUND

Patients with critical illness and respiratory insufficiency may be supported through a variety of means that augment native lung function, such as high-flow nasal cannula, conventional mechanical ventilation, high frequency oscillatory ventilation, and others. Within each of these approaches exists a spectrum of support (i.e., settings) that can be titrated to meet changing patient conditions. Patients may also be supported through other interventions, such as though a dose of medication prescribed to alleviate one or more of the patient's symptoms. Each individual patient may respond differently to a particular medication or dosage.

### SUMMARY

In general, in one aspect, this document features a method for determining a respiratory support score. In the method, operating parameters of one or more respiratory support devices associated with a patient are received at one or more processing devices, and based on the operating parameters, a respiratory support type associated with the patent is determined. A corresponding score range for the respiratory support type is obtained, a respiratory support score within the corresponding score range is determined based on at least a subset of the operating parameters representative of intra-range variation within the corresponding score range. The respiratory support score is presented on a user interface as part of a time-series of respiratory support scores for the patient.

In some implementations, the respiratory support score can be determined to satisfy a threshold condition, and an alert indicative of the respiratory support score can be generated in response. In some cases, patient clinical data can be overlaid on the time-series of respiratory support scores in the user-interface such that the patient clinical data shares the same time-series as the respiratory support scores.

In some cases, a time point corresponding to a clinical action performed on the patient can be identified, parameters indicative of a clinical condition of the patient can be tracked for a time range that includes the time point corresponding to the clinical action, the clinical condition expected to be affected by the clinical action, and changes to the parameters over the time range can be represented on the user-interface with respect to the time-series of the respiratory support scores. In some cases, a metric indicative of an efficacy of the clinical action performed on the patient can be determined based on the parameters tracked over the time range, and the metric can be presented in the user-interface with respect to the time-series of the respiratory support scores. In some cases, the clinical action can include administering a drug at a particular dosage. In some cases, an end point of the time range can be determined based on an expected time for the drug at the particular dosage to have a target effect. In some cases, the parameters tracked over the time range can include at least one of: heart rate, blood pressure, venous pressure, oxygen saturation, and pain level.

In another aspect, a system includes memory and one or more processing devices. The one or more processing devices are configured to obtain operating parameters of one or more respiratory support devices associated with a patient, and determine, based on the operating parameters, a respiratory support type associated with the patient. The one or more processing devices are also configured to obtain a corresponding score range for the respiratory support type, and determine, based on at least a subset of the operating parameters, a respiratory support score in the corresponding score range. The subset of the operating parameters is representative of intra-range variation within the corresponding score range. The one or more processing devices are further configured to present, on a user-interface, the respiratory support score as a part of a time-series of respiratory support scores for the patient.

In some implementations, the storage medium of the system can include additional instructions that, when executed by the processor, cause the processor to determine that the respiratory support score satisfies a threshold condition and, in response, generate an alert indicative of the respiratory support score. In some cases, the additional instructions can cause the processor to present, on the user-interface, patient clinical data, the patient clinical data being overlaid on the time-series of respiratory support scores such that the patient clinical data shares the same time-series as the respiratory support scores.

In some cases, the additional instructions can cause the processor to identify a time point corresponding to a clinical action performed on the patient, track parameters indicative of a clinical condition of the patient for a time range that includes the time point corresponding to the clinical action, the clinical condition expected to be affected by the clinical action, and represent, on the user-interface, changes to the parameters over the time range with respect to the time-series of the respiratory support scores. In some cases, the additional instructions can cause the processor to determine, based on the parameters tracked over the time range, a metric indicative of an efficacy of the clinical action performed on the patient and present the metric on the user-interface with respect to the time-series of the respiratory support scores. In some cases, the clinical action can include administering a drug at a particular dosage. In some cases, an end point of the time range can be determined based on an expected time for the drug at the particular dosage to have a target effect. In some cases, the parameters tracked over the time range can include at least one of: heart rate, blood pressure, venous pressure, oxygen saturation, and pain level.

In another aspect, this document features one or more machine-readable storage devices having encoded thereon computer readable instructions for causing one or more processing devices to perform various operations. The operations include obtaining operating parameters of one or more respiratory support devices associated with a patient, and determining, based on the operating parameters, a respiratory support type associated with the patient. The operations include obtaining a corresponding score range for the respiratory support type, and determining, based on at least a subset of the operating parameters, a respiratory support score in the corresponding score range. The subset of the operating parameters is representative of intra-range variation within the corresponding score range. The operations also include presenting on a user-interface, the respiratory support score as a part of a time-series of respiratory support scores for the patient.

In some implementations, the machine-readable storage device can include additional computer readable instructions for causing the processing device to determine that the respiratory support score satisfies a threshold condition and, in response, generate an alert indicative of the first respiratory support score. In some cases, the additional computer-readable instructions can cause the processor to present, on the user-interface, patient clinical data, the patient clinical data being overlaid on the time-series of respiratory support scores such that the patient clinical data shares the same time-series as the respiratory support scores.

In some cases, the machine-readable storage device can include additional computer readable instructions for causing the processing device to identify a time point corresponding to a clinical action performed on the patient, track parameters indicative of a clinical condition of the patient for a time range that includes the time point corresponding to the clinical action, the clinical condition expected to be affected by the clinical action, and represent, on the user-interface, changes to the one or more parameters over the time range with respect to the time-series of the respiratory support scores. In some cases, the machine-readable storage device can include additional computer readable instructions for causing the processing device to determine, based on the parameters tracked over the time range, a metric indicative of an efficacy of the clinical action performed on the patient and present the metric on the user-interface with respect to the time-series of the respiratory support scores. In some cases, the clinical action can include administering a drug at a particular dosage. In some cases, an end point of the time range can be determined based on an expected time for the drug at the particular dosage to have a target effect. In some cases, the one or more parameters tracked over the time range can include at least one of: heart rate, blood pressure, venous pressure, oxygen saturation, and pain level.

Implementations may include one or combination of two or more of the following features.

The operating parameters can be obtained from one or more electronic medical records of the patient or medical devices associated with the patient. The operating parameters can be obtained from the one or more respiratory support devices. The operating parameters can include at least one of fraction of inspired oxygen (FiO2), nasal cannula flow rate, aerosol mask flow rate, positive end-expiratory pressure (PEEP), positive inspiratory pressure (PIP), pressure support (PS), mandatory rate, set, mandatory and spontaneous tidal volume, high frequency oscillator ventilator (HFOV) frequency, HFOV amplitude, mean airway pressure, high frequency jet ventilator (HFJV) PEEP, HFJV PIP, HFJV rate, extracorporeal membrane oxygen (ECMO) time, ECMO flow, and amount of inhaled nitric oxide. The operating parameters can include at least one of a bilevel positive airway pressure (BiPAP) identifier; a continuous positive airway pressure (CPAP) identifier; a ventilator mode (VM) identifier, an extracorporeal membrane oxygen (ECMO) type identifier; an identifier of the patency of an artificial airway; an identifier of the use of an artificial airway; and a respiratory support device identifier.

The corresponding score range for the respiratory support type can depend on an age of the patient or on a clinical condition of the patient. The user-interface can identify intubation and extubation time points with respect to the time-series of the respiratory support scores. The user-interface can identify a location of the patient with respect to the time-series of the respiratory support scores.

Determining the respiratory support type can include identifying that at least two of the operating parameters provide conflicting information usable in determining the RST, identifying at least a second respiratory support type corresponding to a time point within a threshold distance in the time series, and determining the respiratory support type to be same as the second respiratory support type. The respiratory support type can be one of a room air, nasal cannula, aerosol mask, high-flow nasal cannula (HFNC), continuous positive airway pressure (CPAP), bilevel positive airway pressure (BiPAP), pressure support (PSV), pressure control ventilation (PCV), pressure support (PS), volume control ventilation (VCV) high frequency oscillator ventilator (HFOV), high frequency jet ventilator (HFJV), and venoarterial or venovenous extracorporeal membrane oxygen (ECMO).

In general, in another aspect, this document describes a method for determining a medication efficacy score. The method includes identifying a time point corresponding to a clinical action performed on a patient, and tracking one or more parameters indicative of a clinical condition of the patient for a time range that includes the time point corresponding to the clinical action. The clinical action includes an administration of a drug at a particular dosage. Based on the parameters tracked over the time range, a metric indicative of an efficacy of the clinical action performed on the patient is determined. The metric is presented on a user-interface with respect to a time-series of clinical actions for the patient.

In another aspect, a system includes memory and one or more processing devices. The one or more processing devices are configured to identify a time point corresponding to a clinical action performed on a patient, the clinical action including an administration of a drug at a particular dosage, and track parameters indicative of a clinical condition of the patient for a time range that includes the time point corresponding to the clinical action. The one or more processing devices are also configured to determine, based on the one or more parameters tracked over the time range, a metric indicative of an efficacy of the clinical action performed on the patient, and present, on a user-interface, the metric with respect to a time-series of clinical actions for the patient.

In another aspect, this document features one or more machine-readable storage devices having encoded thereon computer readable instructions for causing one or more processing devices to perform various operations. The operations include identifying a time point corresponding to a clinical action performed on a patient, the clinical action including an administration of a drug at a particular dosage, and tracking parameters indicative of a clinical condition of the patient for a time range that includes the time point corresponding to the clinical action. The operations further include determining based on the one or more parameters tracked over the time range, a metric indicative of an efficacy of the clinical action performed on the patient, and presenting, on a user-interface, the metric with respect to a time-series of clinical actions for the patient.

Implementations may include one or combination of two or more of the following features.

An end point of the time range can be determined based on an expected time for the drug at the particular dosage to have a target effect. The parameters tracked over the time range can include at least one of: heart rate, blood pressure, oxygen saturation (SpO2), and pain level. Determining the metric can include applying a linear regression to the one or more parameters tracked over time.

These and other aspects, features, and implementations can be expressed as methods, apparatus, systems, components, program products, methods of doing business, means or steps for performing a function, and in other ways, and will become apparent from the following descriptions, including the claims.

### DESCRIPTION OF DRAWINGS

FIG. 1 is a block diagram of an example electronic medical record platform.

FIG. 2 is an example of a user-interface presenting visualization of a patient's respiratory support score (RSS) over time.

FIG. 3 is a linear regression graph of a patient's heart rate.

FIG. 4 is a graph of presumed percent of peak effect.

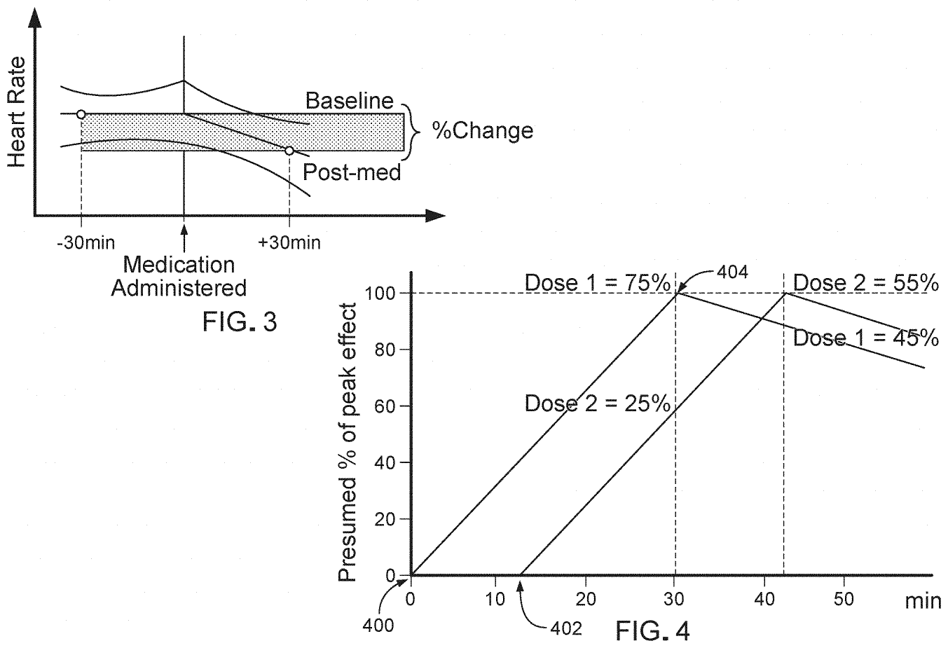

FIG. 5 is an example of a user-interface presenting visualization of a patient's dosage history and medication efficacy score (MES) over time.

FIG. 6 is a flowchart of an example process for calculating a RSS.

FIG. 7 is a flowchart of an example process for calculating a MES.

FIG. 8 is a schematic diagram of a computer system.

Like reference symbols in the various drawings indicate like elements.

### DETAILED DESCRIPTION

The present disclosure describes technology that provides for the creation of a respiratory support score (RSS) that summarizes a patient's entire spectrum of respiratory support at any given point in time. In addition, the present disclosure provides for a medication efficacy score (MES) that identifies the effectiveness of a given medication dosage for a particular patient. Each of these scores may provide several benefits that can improve patient care quality, increase healthcare efficiency, decrease medical errors, facilitate health communication, and enable better caregiver decision-making, among others.

Although its causes are diverse, respiratory insufficiency is a common feature of critically ill patients. Thus, respiratory support represents an important marker of patient wellness. Currently, no summarial score exists to summarize the spectrum of respiratory support provided to patients over time. For example, caregivers may describe that the mandatory ventilator rate was weaned, that a patient was transitioned to pressure support-only ventilation, or that a patient was extubated from conventional ventilator to CPAP, but may not be able to summarize the spectrum of respiratory support provided to the patient.

The RSS can facilitate visualization of a patient's trajectory over their course of critical illness. Because respiratory support is a common component of intensive care, it can provide a roadmap for the support that a patient has received during their hospital stay. For instance, decreases in the RSS may be a sign that a patient is improving along an expected trajectory, while the converse may imply a worsening patient condition and may permit early identification of changes in the patient's respiratory status or predict extubation failure. Similarly, the RSS may identify when a patient's ventilator weaning trajectory has stalled, which may prompt clinical re-evaluation. Importantly, the RSS summarizes features of the care provided, rather than the patient's response to it.

By enabling the visualization of the entire spectrum of a patient's respiratory support over the course of treatment, the RSS can provide caregivers with information that would otherwise be obfuscated in multiple medical records across disparate data sources and user-interfaces. This in turn allows caregivers to efficiently access information needed to make more informed medical decisions and reduces the chance of medical error. The RSS may also permit early identification of trends in a patient's degree of respiratory support that may be difficult to detect through analysis of individual variables (e.g., the mandatory ventilation rate and peak inspiratory pressure change minimally, but viewed within the context of one another the change may be more visible).

Further, predictive information may exist in a patient's RSS over time. Tabulating the RSS over time may permit quantification of a patient's burden of respiratory illness and predict future length of stay, or visualizing the slope of the score during weaning may predict the likelihood of successful extubation.

The RSS may also improve patient care quality by permitting comparison of respiratory support weaning practices between and within patient cohorts in a single ICU and across centers. The RSS may provide a construct within which to quantify the variability in ventilator weaning trajectories for specific patient populations, such as patients following a particular operation or with a specific diagnosis, thereby improving care quality. The RSS can also improve care quality and hospital efficiency by providing a means to rank which patients need the most attention in a busy environment.

Because each manually-recorded EMR element sustains a small but defined fraction of error, the use of logic against multiple variables, entered over time by multiple providers, allows the RSS and other derived variables to cancel out the effect of such errors in the final value. When a potential error is detected, or an abnormal RSS is produced, the caregiver may be alerted to prompt resolution. Further, the concept of the derived variable that is based on a defined logic applied to previously defined data elements may enhance the accuracy and efficiency of recording these fields for institutional reporting to quality databases.

In addition to the benefits described above, the MES can facilitate visualization of an individual patient's response to a particular medication and dosage. In general, each dose of medication, such as pain medication, has an intended effect (e.g., to decrease the patient's signs and symptoms of pain). However, each individual patient may respond differently to a particular medication or dosage. Currently, data regarding a medication's administration and its effects on a patient cannot be visualized contemporaneously or formally analyzed. For example, vital signs such as heart rate, respiratory rate, and blood pressure are displayed on the bedside monitor. Medication dose and time of medication administration are displayed in the medical administration record (MAR). A patient's sedation score is documented in yet another separate portion of nursing documentation in the patient's EMR. By combining data from these disparate sources and providing feedback regarding a patient's response to each dose of medication, the MES can allow caregivers to make a more informed decision on an appropriate prescription plan for the individual patient. In doing so, the MES can enable personalized care that is safer and more effective for the patient. Furthermore, the MES can allow caregivers to compare the efficacy of medications across similar patient populations which can establish best practices and improve patient care quality.

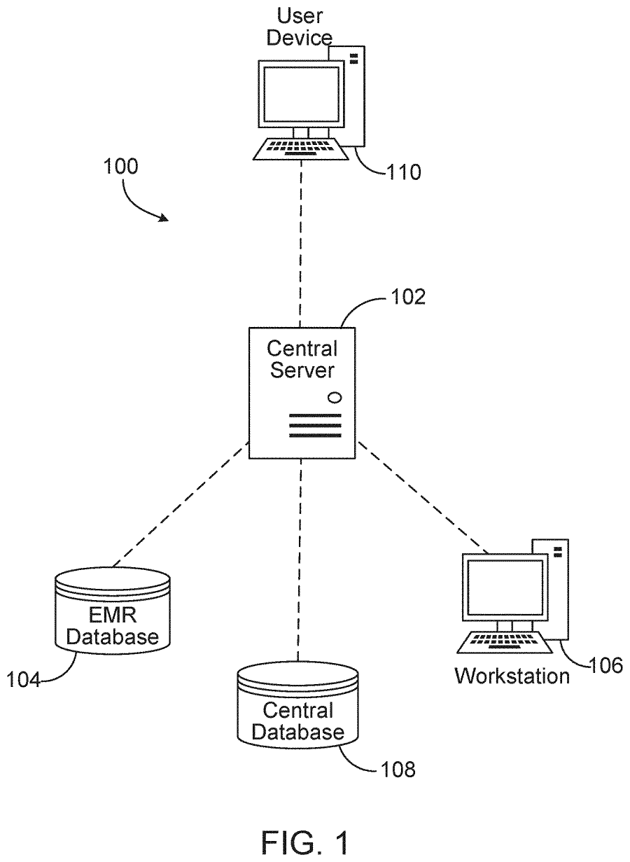

FIG. 1 illustrates an electronic medical record (EMR) platform 100 configured to carry out various aspects of the present disclosure. The EMR platform 100 can include a central server 102 in communication with one or more EMR databases (or data streams) 104 to securely receive patient data, such as medication data, order data, lab data, equipment settings data, diagnoses, and vital signs, among others. In some implementations, the central server 102 may also receive data from a user-interface provided on one or more point of care workstations 106, or from ventilation devices or other medical devices associated with the patient. The retrieved data can be securely stored in a central database 108 for processing by the central server 102. For example, the central server 102 can process the retrieved data to determine a respiratory support score (RSS) and/or a medication efficacy score (MES) for a particular patient, as described herein. The central server 102 can present the processed data to a user, such as a caregiver, through an interactive user-interface provided on the workstation 106 or a user device 110, which may be a desktop, laptop, tablet, wearable device, or smartphone, among others.

To determine the RSS for a particular patient, the central server 102 may derive one or more variables to supplement or aid in the calculation of the RSS. In general, derived variables are clinically meaningful variables whose values are not recorded directly within a specific field of a patient's EMR, but which can be accurately derived by, for example, applying a string of logic to the fields within one or more EMR or otherwise interfacing with the workstation 106 or other equipment at the point of care. One such variable is the respiratory support type (RST). The central server 102 may identify the RST associated with the patient, for example, during each minute of the patient's hospitalization. The RST can indicate the type of respiratory support being provided to the patient, such as extracorporeal membrane oxygen (ECMO) life support, high frequency jet ventilation (HFJV), high frequency oscillatory ventilation (HFOV), mechanical ventilation, pressure support ventilation, biphasic positive airway pressure (BiPAP), continuous positive airway pressure (CPAP), high flow nasal cannula (HFNC), aerosol mask, nasal cannula, and room air, among others. The RST can also indicate situations where the patient is receiving no respiratory support.

As noted above, the RST can be derived by reference to one or more fields within the patient's EMR. A logic string can be used to identify the RST based on the recorded data elements in the patient's EMR that would define (“defining”), be consistent with (“allowable”), or be impossible with (“conflicting”) a particular RST. As a non-limiting example, a list of defining, allowable, and conflicting data entries within each of the identifying data fields associated with each RST is identified in Table 1. For example, an entry of ‘Pressure Support Ventilation’ (PSV) in a ‘Ventilator Mode’ field of the patient's EMR may be considered as defining the RST as pressure support ventilation. The same entry may also be considered as conflicting with any other RST (e.g., if a ‘2’ was erroneously entered in a “Nasal Cannula Flow” field, suggesting that the patient was simultaneously on pressure support ventilation and nasal cannula, then the algorithm may consider that internally conflicting data). Similarly, an entry of ‘Endotracheal Tube’ within an ‘Airway Assessment’ field may be considered allowable for any invasive mode of ventilation, but conflicting for any non-intubated mode of ventilation. Using this approach, all of the relevant data within the patient's EMR can be considered in a tiered fashion to determine the minimum number of variables necessary for assigning an RST to the patient. In other implementations, the RST can be received directly, for example, from an input to an application running on the workstation 106.

TABLE 1
List of defining, allowable, and conflicting data entries within each of the key identifying data
fields associated with each RST in accordance with one embodiment.

| Field Header | Possible field entries | Defining for | Allowable for |
|---|---|---|---|
| Airway | Tracheostomy | | All RSTs: RA, BB, NC, MASK, |
| Assessment | | | HFNC, CPAP, BiPAP, PSV, |
| | | | PCV, VCV, HFOV, HFJV, |
| | | | ECMO |
| | Endotracheal | | PSV, PCV, VCV, HFOV, |
| | tube | | HFJV, ECMO |
| | No compromise | | All RSTs: RA, BB, NC, MASK, |
| | | | HFNC, CPAP, BiPAP, PSV, |
| | | | PCV, VCV, HFOV, HFJV, |
| | | | ECMO |
| Oxygen | None | RA | ECMO |
| Delivery | Blow by | Blow by | NC, MASK, HFNC, CPAP, |
| Device: FiO2 | | | BiPAP, ECMO |
| | Aerosol mask | MASK | ECMO |
| | Face tent | MASK | ECMO |
| | High-flow | HFNC | ECMO |
| | nasal cannula | | |
| | BiPAP | BiPAP | ECMO |
| | CPAP | CPAP | ECMO |
| | Ventilator | | BiPAP, CPAP, PSV, PCV, |
| | | | VCV, HFOV, HFJV, ECMO |
| Oxygen Delivery | None | RA | ECMO |
| Device: Liters | Blow by | Blow by | NC, MASK, HFNC, CPAP, |
| per minute | | | BiPAP, ECMO |
| | Nasal cannula | NC | ECMO |
| | High-flow | HFNC | ECMO |
| | nasal cannula | | |
| | BiPAP | BiPAP | ECMO |
| | CPAP | CPAP | ECMO |
| Oxygen Source | Room air | RA | ECMO |
| | Oxygen delivery | | NC, Blow by, MASK, HFNC, |
| | device | | CPAP, BiPAP, PSV, PCV, VCV, |
| | | | HFOV, HFJV, ECMO |
| Ventilator Mode | CPAP | CPAP | ECMO |
| | BiPAP | BiPAP | ECMO |
| | NIPPV | BiPAP | ECMO |
| | NPPV-PSV | BiPAP | ECMO |
| | PSV | PSV | ECMO |
| | Pressure-A/C | PCV | ECMO |
| | PCV-SIMV + PSV | PCV | ECMO |
| | PCV | PCV | ECMO |
| | PRVC | VCV | ECMO |
| | PRVC-SIMV | VCV | ECMO |
| | VCV-SIMV + PSV | VCV | ECMO |
| | VCV | VCV | ECMO |
| | HFOV 3100-A | HFOV | ECMO |
| | HFOV 3100-B | HFOV | ECMO |
| EtCO2 | 5-100 | PSV, PCV, VCV | ECMO |
| FiO2 | 0.21-1 | | All RSTs: RA if = 0.21 or 0, BB, |
| | | | NC, MASK, HFNC, CPAP, |
| | | | BiPAP, PSV, PCV, VCV, |
| | | | HFOV, HFJV, ECMO |
| Nasal cannula flow | 0.125-4 | | BB, NC, HFNC, MASK, ECMO |
| High Flow nasal | February-40 | HFNC | MASK, ECMO |
| cannula flow | | | |
| CPAP PEEP | 15-Mar | CPAP | ECMO |
| BiPAP EPAP | 15-Mar | | CPAP, BiPAP, ECMO |
| BiPAP IPAP | 0-42 | BiPAP | ECMO |
| BiPAP Rate | April-45 | BiPAP | ECMO |
| PEEP | 15-Mar | | CPAP, BiPAP, PSV, PCV, VCV, |
| | | | HFJV, ECMO |
| PIP | August-40 | | BiPAP, PSV, PCV, VCV, HFJV, |
| | | | ECMO |
| PS | 25-May | | BiPAP, PSV, PCV, VCV, HFJV, |
| | | | ECMO |
| Ventilator rate | May-50 | | BiPAP, PCV, VCV, HFJV, |
| | | | ECMO |
| Tidal volume set | 15-Mar | VCV | ECMO |
| (ml/kg) | | | |
| Tidal volume | 15-Mar | | PSV, PCV, VCV, ECMO |
| spontaneous | | | |
| (ml/kg) | | | |
| Tidal volume | 15-Mar | | PSV, PCV, VCV, ECMO |
| mandatory | | | |
| (ml/kg) | | | |
| HFJV PEEP | 15-Apr | HFJV | ECMO |
| HFJV PIP | September-50 | HFJV | ECMO |
| HFJV rate | 300-480 | HFJV | ECMO |
| HFOV amplitude | 15-80 | HFOV | ECMO |
| HFOV hertz | 15-May | HFOV | ECMO |
| HFOV mean | 25-Mar | HFOV | ECMO |
| airway pressure | | | |
| ECMO Flow | 0-150 | ECMO | |
| (ml/kg/min) | | | |

Abbreviations: FiO2: fraction of inspired oxygen, CPAP: continuous positive airway pressure, BiPAP: bilevel positive airway pressure, PEEP: positive end-expiratory pressure, PIP: positive inspiratory pressure, PS: pressure support, PCV: pressure control ventilation, VCV (volume control ventilation), HFOV: high frequency oscillator ventilator, HFJV: high frequency jet ventilator, OS: oxygen source, ODD: oxygen delivery device, AA: airway assessment, VM: ventilator mode.

The central server 102 may also collect numerical and/or non-numerical settings data associated with the respiratory support device. The central server 102 may derive these settings by querying the relevant EMR database 104, applying a logic string to the patient's EMR, or otherwise searching through the patient's EMR. In some cases, one or more fields of the EMR can identify the patency and use of an artificial airway or a respiratory support device, among others. In some implementations, the central server 102 may be configured to derive certain settings based on the RST, as shown in Table 2. For example, if the RST is determined to be high flow nasal cannula (HFNC), then the flow rate can be derived to quantify the level of ventilatory support. In other cases, if the RST is determined to be pressure support, then the positive-end expiratory pressure (PEEP) can be derived to quantify the level of ventilatory support.

TABLE 2
The settings variables derived for each RST in accordance with one embodiment.

| Respiratory support type | Numerical Fields | Non-Numerical Fields |
|---|---|---|
| Room air | FiO2 | Oxygen Source (OS), Oxygen |
| | | Delivery Device (ODD), |
| | | Airway Assessment (AA) |
| Nasal cannula | Flow rate, FiO2 | OS, ODD, AA |
| Aerosol mask | Flow rate, FiO2 | OS, ODD, AA |
| High flow nasal cannula | Flow rate, FiO2 | OS, ODD, AA |
| CPAP | PEEP, FiO2 | OS, ODD, AA, BiPAP_CPAP, |
| | | Ventilator Mode (VM) |
| BiPAP | PEEP, PIP, rate, FiO2 | OS, ODD, AA, BiPAP_CPAP, VM |
| Pressure support (PSV) | PEEP, PIP, PS, FiO2 | OS, ODD, AA, VM |
| PCV: SIMV-PC/PS | PIP, PEEP, mandatory rate, | OS, ODD, AA, VM |
| | mandatory and spontaneous tidal | |
| | volume, PS, FiO2 | |
| VCV: SIMV-VC/PS | PIP, PEEP, mandatory | OS, ODD, AA, VM |
| | rate, set, mandatory and | |
| | spontaneous tidal volume, | |
| | PS, FiO2 | |
| HFOV | Frequency, amplitude, | OS, ODD, AA, VM |
| | mean airway pressure, FiO2 | |
| HFJV | HFJV PEEP, HFJV PIP, | OS, ODD, AA, VM |
| | HFJV rate, PIP, PEEP, FiO2 | |
| ECMO (venoarterial and | ECMO hour, ECMO flow | ECMO type |
| venovenous) | | |

In some cases, the settings data is manually entered into the EMR when there are changes in care, changes in settings within a single RST, or changes in respiratory support types. Moreover, in some cases, only the change being made is recorded when the settings are changed within a single RST (e.g., when the ventilator rate is weaned, only the new ventilator rate is recorded at a single timestamp without any of the other ventilator settings). Although this practice may be sufficient for patient care, it may be preferable for data warehousing and analysis that all available settings data be derived for each time point. Thus, where there is missing or conflicting data, a confidence score may be assigned to the RST and the associated data to identify the quality of the data at that timestamp. For example, a confidence score of 1 or 2 can reflect the presence of at least one defining field for the RST and no conflicting fields. A confidence score of 3 can reflect timestamps where only allowable data elements were present in the absence of a defining data element. In these time points, temporally neighboring data elements can be examined; if one or more adjacent RST are not in conflict with the current row, the RST can be imputed into that timepoint. A confidence score of 4 can be assigned in the presence of a conflict in which there were simultaneous entries of defining or allowable fields for disparate RSTs. In these cases, neighboring timepoints can be examined; if conflict is resolved (e.g., if the RSTs are the same before and after the timepoint), then the RST can be defined with a confidence score of 4. In other cases of conflicting data, the timestamp and its associated data can be removed. A summary of one implementation of the confidence score logic is provided in Table 3.

TABLE 3
Summary of confidence score logic in accordance with one embodiment.

| | | | | Neighboring | |
|---|---|---|---|---|---|
| Score | Defining | Allowed | Conflicting | rows | Action |
| 1* | Must be | May be | Not present | Not evaluated | RST assigned |
| | present | present | | | |
| 2* | Must be | May be | Not present | Not evaluated | RST assigned |
| | present | present | | | |
| 3 | Not | Must be | Not present | Surrounding | RST assigned |
| | present | present | | RST is not in | |
| | | | | conflict with | |
| | | | | current row | |
| 4 | Not | May be | Common | RST from | RST imputed |
| | present | present | error | timepoint before | from |
| | | | pattern | and after are the | neighboring |
| | | | present | same | timepoints |
| NA | Not | Not | Heavy | Not evaluated | Delete timepoint |
| | present | present | conflict | | |
| | | | present | | |

*Confidence scores 1 and 2 can differ by the amount of defining data present at a timestamp.

In some implementations, the central server 102 can derive one or more additional variables to verify the assigned RST and/or its associated data. For example, in some cases, the server can verify the RST by deriving intubation and extubation endpoints. A timestamp can be defined as extubation if, for example, there is a change from an intubated RST to two or more consecutive timestamps with a non-intubated RST. Conversely, a timestamp can be defined as intubation where there is an identified reintubation using a non-intubated RST followed by two or more consecutive timestamps with an intubated RST. In some cases, derivation of the intubation and/or extubation endpoints can require that the RST assignment have a certain confidence score, such as 1 or 2, for the timestamp to be considered. Deriving the intubation and extubation endpoints can also allow the server to determine the frequency and duration of intubation or reintubation.

In order to classify reintubations as procedural or non-procedural, the central server 102 may identify all cardiac and non-cardiac procedures during the relevant hospital stays by a particular patient. Each of these procedures can be identified by querying the relevant EMR database 104, applying a logic string to the patient's EMR, or otherwise searching through the patient's EMR. A primary procedure can be identified as the first surgical procedure the patient received after hospital admission. Alternatively, a primary diagnosis may be the one which is associated with a majority of interventions in a patient's hospital record. All other cardiac and non-cardiac interventional and surgical procedures can be considered a re-operation. Procedural reintubations can be identified as procedures in which a patient was not intubated prior to the procedure but was ventilated following the procedure stop time. If a patient was reintubated for a procedure prior to the procedure start time, then the reintubation can be identified as a non-procedural reintubation.

After deriving the RST and various other values, the central server 102 can calculate the RSS for a particular patient. In general, the RSS may be an empirically weighted score ranging from a minimum respiratory support score (e.g., 0) to a maximum respiratory support score (e.g., 100). In some implementations, this range can be subdivided into one or more sub-ranges, with each sub-range representing an individual RST or multiple, equivalent RSTs. Each sub-range can define an upper RSS limit and a lower RSS limit for the corresponding RST or group of RSTs.

The settings and other data that describe the amount of ventilatory support delivered for a given RST can be identified, as described above, and the amount of ventilatory support that each setting provides within the corresponding RST may be quantified. For example, the central server 102 may assign an upper and lower limit to each setting representing a maximum and minimum amount of ventilatory support that the setting can provide within the corresponding RST. From here, the server can use the current value of each setting to quantify the amount of ventilatory support it provides in proportion to the assigned upper and lower limits. In some cases, the assigned limits can be varied based on one or more patient attributes, such as patient age, patient gender, clinical condition, or disease type, among others. Additionally, each setting can be weighted to represent its overall contribution to the amount of ventilatory support for a particular RST. For example, the mandatory rate may be the primary means by which most providers wean the ventilator, so this may be weighted more heavily than, for example, inspiratory time or PEEP. Notably, for some variables, an increase in value represents a decrease in respiratory support (e.g., the frequency on HFOV is decreased in order to increase CO2 clearance). To account for this, the effect of such variables on the RSS can be inverted.

To calculate the RSS, each ventilatory support setting for a given RST can be quantified in proportion to its assign upper and lower limits and weighted empirically. The quantified value of each setting can then be summed and multiplied by the RSS sub-range assigned to the RST to create a differential RSS value. The differential RSS value can be added to the base RSS value for the corresponding RST to determine the RSS. The calculation of the RSS is summarized in equation (1), and an example calculation is provided in Table 4. The RSS can be associated with a particular timestamp, which may be normalized to the date and time of the patient's index cardiac operation for each hospitalization.

$$RSS = {RST}_{base} + {RST}_{sub \text{-} range} [ \sum [ ( \frac{{Setting}_{current} - {Setting}_{min}}{{Setting}_{max} - {Setting}_{min}} ) \cdot ( Setting weight ) ] ] \qquad ( 1 )$$

TABLE 4
Example RSS calculation in accordance with one embodiment.
Example RSS Calculation

| Patient age | 3 weeks |
|---|---|
| Ventilator settings | SIMV PC-PS; PIP 28 cm H2O; PEEP 8 cm H2O; rate 24/min; |
| | PS 8 cm H2O; FiO2 40% |
| ETT size | 3.5; PSVmin = 10 cm H2O |
| RST assignment | Conventional mechanical ventilation |
| Points assigned for RST assignment | 36 |
| PIP (5%) | PIP range for age: 15-30 cm H2O |
| | Patient PIP = 28 cm H2O |
| | PIP settings points (unweighted) = (28 − 15)/(30 − 15) = 0.93 |
| | PIP settings points (weighted) = 0.93*0.05 = 0.0465 |
| PEEP (5%) | PEEP range for age: 5-10 cm H2O |
| | Patient PEEP = 8 cm H2O |
| | PEEP settings points (unweighted) = (8 − 5)/(10 − 5) = 0.6 |
| | PEEP settings points (weighted) = 0.6*0.05 = 0.03 |
| Rate (75%) | Rate range for age: 10-28/min |
| | Patient Rate = 24/min |
| | Rate settings points (unweighted) = (24 − 10)/(28 − 10) = 0.78 |
| | Rate settings points (weighted) = 0.78*0.75 = 0.585 |
| FiO2 (5%) | FiO2 range: 21-100% |
| | Patient FiO2 = 40% |
| | FiO2 settings points (unweighted) = (40 − 21)/(100 − 21) = 0.24 |
| | FiO2 settings points (weighted) = 0.24*0.05 = 0.012 |
| PS (5%) | PS range for age: 10 (PSVmin)-14 cm H2O |
| | Patient PS = 8 cm H2O |
| | PS settings points (unweighted) = (10 − 10)/(14 − 10) = 0* |
| | PS settings points (weighted) = 0*0.05 = 0 |
| Total settings points | 0.0465 + 0.03 + 0.585 + 0.012 + 0 = 0.6735 |
| Weighted settings | Max − min of RST × total settings points = (70 − 36) (0.6735) = 22.9 |
| points for CMV | |
| RSS | 36 + 22.9 = 58.9 |

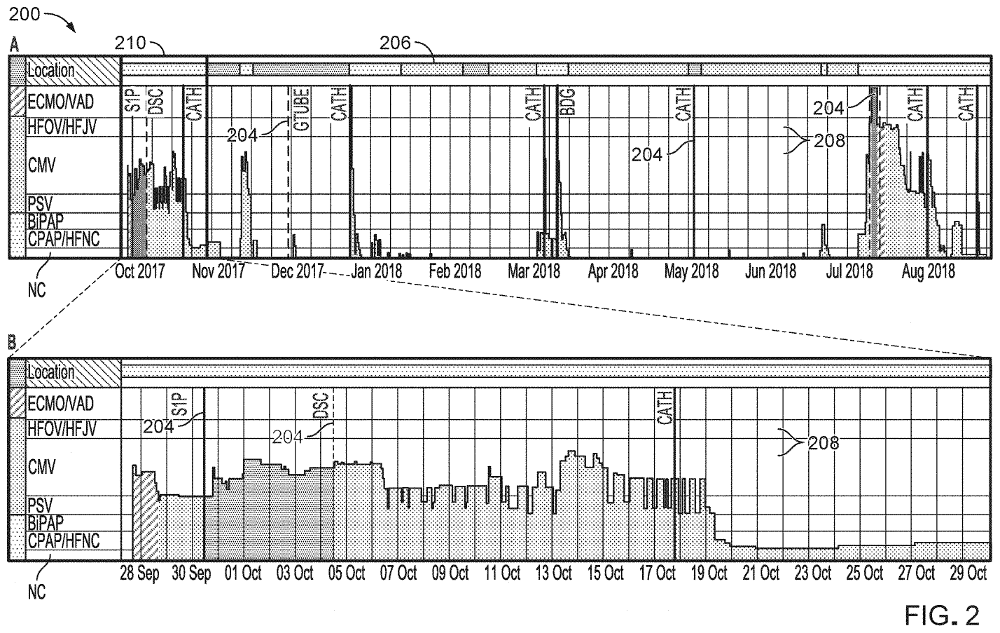

Referring to FIG. 2, the central server 102 can provide a user-interface 200 to visualize the resulting RSS as a function of time for a particular patient. The user-interface 200 can be presented on the user device 110 or the workstation 106, for example, using a native or web-based application. In doing so, the user-interface 200 can provide a tool that visually links and analyzes patient data in a way that makes it easy for a caregiver to understand, thereby improving caregiver decision-making and patient care.

As shown in FIG. 2, the user-interface 200 can include a graph that depicts a patient's RSS 202 (vertical axis) over time (horizontal axis). Overlaid on the graph are indicators 204 for various medical events, such as treatment with paralytic agents, inhaled nitric oxide (iNO), stage 1 palliation, ancillary procedures, cardiac catheterizations, echocardiograms, and discharge, among others. In some cases, the patient's location 206, such as home, floor, or ICU, is shown on the timeline. The vertical axis of one or more graphs in the user-interface 200 can be divided into segments 208 to indicate how the RSS 202 was subdivided into intervals representing an individual RST or multiple, equivalent RSTs. This feature can aid the user in identifying instances where a patient was under a minimal amount of ventilatory support for a particular RST. In some implementations, various aspects of the user-interface 200 can be color-coded to facilitate readability and ease of understanding.

In some implementations, the user-interface 200 can be interactive to improve the user experience and enable the user to gain more information. For example, the user-interface 200 can include a movable window 210 that enables a user to select a portion of the RSS data (e.g., in RSS graph “A”) to be displayed in more detail, such as in a second RSS graph “B”. In some cases, key findings 212 associated with a specific RSS or medical event may be displayed on hover over, and reports, include discharge summaries, may be available on click. A click or other user interaction may also provide an automated comparison 214 of data, such as heart rate, blood pressure, venous pressure, oxygen saturation, pain level, among others, prior to 216 and following 218 an event 220, such as a spontaneous breathing trial. In some cases, a regression 222, such as a linear regression, may be applied to the data, and a key metric 224, such as a median or a Z score, can be indicated.

The user-interface 200 can also time align or overlay the RSS on other data, such as fluid intake/output, nutrition, sedation, or waste, among others, to facilitate comparisons of clinical events over time and to allow for the treatment or condition of an individual patient to be benchmarked against other patients with the same clinical condition and matched to postoperative time. For example, in some cases the user-interface 200 can time align or overlay the RSS on a plot of the serum creatinine to visualize that creatinine went up during a CPR event or when the patient was on ECMO. In some cases, the central server 102 can derive one or more metrics, such as extubation, intubation, or reintubation frequency and/or duration, from the RSS and can present it to the user via the user-interface 200. In some cases, the central server 102 may use the RSS to determine the timing of ECMO use, extubation, intubation, CPAP, BiPAP, and iNO use for reporting to a central repository for quality reporting.

In some implementations, the central server 102 can issue one or more alerts based on the RSS or related data. For example, the central server 102 can send an alert to a caregiver or another user if the RSS reaches a certain threshold. In some cases, the central server 102 can issue an alert in response to certain trends in a patient's RSS, for example, to notify a caregiver of a likely cause of the change in the RSS. The alert may also notify a caregiver of an event derived from the RSS, such as extubation or reintubation. The alerts can be provided to, for example, the workstation 106 and/or the user device 110 through the user-interface 200 or another means of communication, such as an email, a SMS message, or a push notification, among others.

The central server 102 can also be configured to calculate a medication efficacy score (MES) that identifies the effectiveness of a given medication or medication dosage for a particular patient. In general, all doses of medication have an intended effect that can manifest itself through one or more observable metrics (e.g., blood pressure, venous, pressure, respiratory rate, or State Behavioral Scale (SBS)), patient-reported metrics (e.g., pain score, including Face, Legs, Activity, Cry, Consolability (FLACC) and Individualized Numeric Rating Scale (iNRS)), and/or parent-reported metrics. For example, after administering a dose of morphine, a patient's heart rate is expected to decrease and the patient-reported pain score is expected to improve. Thus, to determine whether a dosage of medication is having the intended effect, the central server 102 can obtain data on the various metrics between two predetermined points in time, or endpoints. In some cases, endpoints can include a pre-dosage time (e.g., 30 minutes prior to dosage) and a post-dosage time (e.g., 30 minutes after dosage). In some implementations, the endpoints can be adjusted based on the medication type to account for the different effect timeline of different medications. The central server 102 can obtain the data from one or more of the EMR database 104, the workstation 106, or the central database 108.

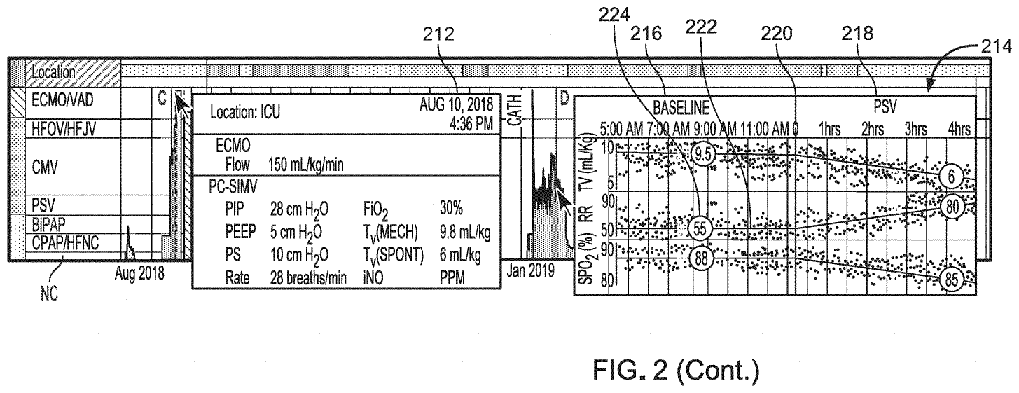

After obtaining the data, the central server 102 can apply a regression, such as a linear regression, to one or more of the metrics. In some implementations, the server can convert the data for one or more of the metrics into Z scores prior to applying the linear regression. For example, in some cases, the data can be converted into age- and/or diagnosis-related Z scores. The server can then establish a baseline value, such as a median Z score, for the predetermined time period prior to the dosage. The slope of the regression line can be used to determine the average change of a particular metric over time following the dose. Moreover, the server can identify the peak effect value as the y-intercept at the predetermined post-dose endpoint, which may represent the estimated time of peak effect. Finally, the central server 102 can calculate the percent change between the baseline value and the peak value, as shown in FIG. 3.

The percent change or other change data for each metric can used to establish the MES for the particular dose, as summarized in Table 5. As shown in Table 5, the MES may range, for example, from 0 to 5, with a score of 0 indicating an ‘ineffective dose,’ or a dose where no statistically significant improvement in any of the metrics occurred. In some implementations, the change data associated with multiple metrics can be combined to determine the MES. In such a case, the change data of certain metrics can be given greater weight when determining the MES.

TABLE 5
Summary of MES assignment in accordance with one embodiment.

| | % Change | Change in Score | |
|---|---|---|---|
| SCORE | (e.g., HR, BR, RR) | (e.g., FLACC, SBS) | Parent Assessment |
| 0 | ≥0 | ≥0 | Did not make child |
| | | | better or worse |
| 1 | −1% to −5% | −1 | Maybe helped a little bit |
| 2 | −6% to −10% | −1 | Helped a bit |
| 3 | −11% to −15% | −2 | Helped a moderate |
| | | | amount |
| 4 | −16% to −20% | −2 | Helped a good amount |
| 5 | ≤−20% | −3 | Helped a lot |

In some cases, multiple doses of medication or different medications are administered in quick succession, which may affect the calculation of the MES. For example, a first dose 400 of a medication may be followed by a second dose 402 of a medication at a time before the first dose 400 reaches its presumed peak effect 404. Accordingly, for instances in which one or more doses of intermittent medications are given prior to the presumed peak effect 404 of the first dose 400, the central server 102 can modify the calculation for the change in Z score such that it is divided between the first dose 400 and the second dose 402 in proportion to the time since administration. To do so, the central server 102 can calculate a dilution constant at the presumed time of peak effect or post-dose endpoint, as summarized in equation (2). The dilution constant can then be applied to the calculated Z score change, percent change, or other change data (e.g., if the first dose 400 is determined to have decreased the Z score by 1, and the dilution constant is calculated to be 0.75, then the first dose a Z score change of 0.75(−1), or −0.75).

$$\% effect of first dose on Z score {change}_{dose x} = \frac{100}{\begin{matrix}{\sum }^{all dose with current effect > 0} \\ presumed peak {effect}_{when dose \times peak reaches 100 \%}\end{matrix}} \qquad ( 2 )$$

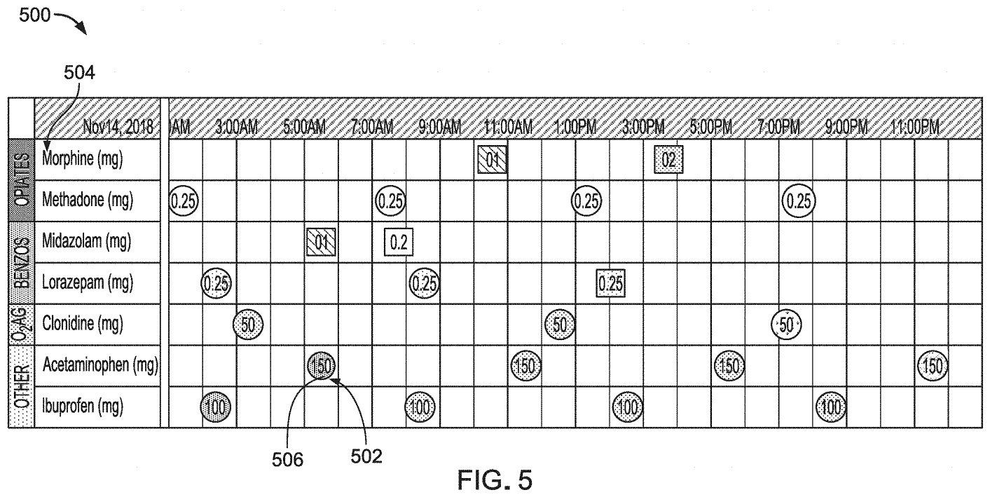

Referring to FIG. 5, the central server 102 can provide a user-interface 500 to visualize one or more medical efficacy scores for a particular patient. The user-interface 500 can be presented on the user device 110 or the workstation 106, for example, using a native or web-based application. In doing so, the user-interface 500 can provide a tool that visually indicates which medications and doses are effective and ineffective for a particular patient, allowing for more personalized and better quality care.

As shown in FIG. 5, the user-interface 500 can include a graph that depicts each dose 502 of a particular medication 504 that a patient has received over time, along with its numerical dosage 506. Each dose 502 can include a particular color or other identifier to enable a caregiver to determine the MES of the dose. In this way, the caregiver or other user of the user-interface 500 can quickly discern which medications and/or doses are effective and ineffective for the particular patient.

In some implementations, the user-interface 500 can select the shape of each dose 502 to indicate the method of administration (e.g., a circular dose icon can represent oral administration, and a square dose icon can represent an intravenous administration). In some cases, similar medications 504 can be grouped by type in the user-interface 500. The user-interface 500 can also flag unusual doses based on the patient's or other patient's dosage history.

The user-interface 500 may be an interactive user-interface. For example, the user-interface 500 can allow a user to interact with a particular dose 502 to gain more information about the dose or its associated MES. In some implementations, the user-interface 500 can enable a user, such as a patient, a parent, or a caregiver, to input their own adjudication of how effective a particular dose of medication was in alleviating symptoms. This input can be fed back into the calculation of the MES for the particular dose 502. The user-interface 500 can also time align other data with the doses 502 to facilitate comparisons of the dose and its associated MES with other clinical event data.

In some implementations, the central server 102 can issue one or more alerts based on the MES or related data. For example, the central server 102 can send an alert to a caregiver or another user, such as a pharmacist, if an unusual dosage or a normal dosage at unusual time is detected. The alerts can be provided to, for example, the workstation 106 and/or the user device 110 through the user-interface 500 or another means of communication, such as an email, a SMS message, or a push notification, among others.

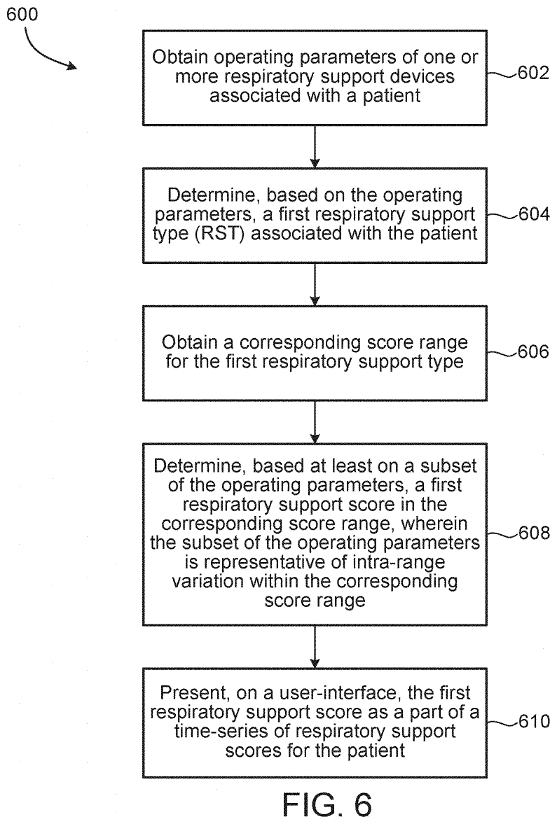

FIG. 6 is a flowchart of an example process 600 for calculating a respiratory support score (RSS). At least a portion of the process 600 can be implemented using one or more processors operation on the central server 102. Operations of the process 600 include obtaining operating parameters of one or more respiratory support devices associated with a patient (602). In some implementations, the operating parameters can be obtained from one or more electronic medical records of the patient, or from the one or more respiratory support devices. The operating parameters can include at least one numerical parameter selected from a group consisting of: fraction of inspired oxygen (FiO2), nasal cannula flow rate, aerosol mask flow rate, positive end-expiratory pressure (PEEP), positive inspiratory pressure (PIP), pressure support (PS), mandatory rate, set, mandatory and spontaneous tidal volume, high frequency oscillator ventilator (HFOV) frequency, HFOV amplitude, mean airway pressure, high frequency jet ventilator (HFJV) PEEP, HFJV PIP, HFJV rate, extracorporeal membrane oxygen (ECMO) time, ECMO flow, and amount of inhaled nitric oxide.

Operations of the process 600 also includes determining, based on the operating parameters, a first respiratory support type (RST) associated with the patient (604). In some implementations, determining the RST can include identifying that at least two of the operating parameters provide conflicting information usable in determining the first RST, identifying at least a second RST corresponding to a time point within a threshold distance in the time series, and determining the first RST to be same as the second RST. The first respiratory support type can include at least one of: room air, nasal cannula, aerosol mask, high-flow nasal cannula (HFNC), continuous positive airway pressure (CPAP), bilevel positive airway pressure (BiPAP), pressure support (PSV), pressure control ventilation (PCV), pressure support (PS), volume control ventilation (VCV) high frequency oscillator ventilator (HFOV), high frequency jet ventilator (HFJV), and venoarterial or venovenous extracorporeal membrane oxygen (ECMO).

In some implementations, the processing at step 604 includes determining that the first respiratory support score satisfies a threshold condition and, in response, generating an alert indicative of the first respiratory support score.

Operations of the process 600 further include obtaining a corresponding score range for the first respiratory support type (606). In some implementations, the corresponding score range for the first respiratory support type depends on an age of the patient or on a clinical condition of the patient.

The process 600 also includes determining, based at least on a subset of the operating parameters, a first respiratory support score in the corresponding score range, wherein the subset of the operating parameters is representative of intra-range variation within the corresponding score range (608).

Operations of the process 600 can further include presenting, on a user-interface, the first respiratory support score as a part of a time-series of respiratory support scores for the patient (610). In some implementations, the user-interface can identify intubation and extubation time points with respect to the time-series of the respiratory support scores. In some cases, the user-interface can identify a location of the patient with respect to the time-series of the respiratory support scores. In some cases, the user-interface can include patient clinical data overlaid on the time-series of respiratory support scores such that the patient clinical data shares the same time-series as the respiratory support scores.

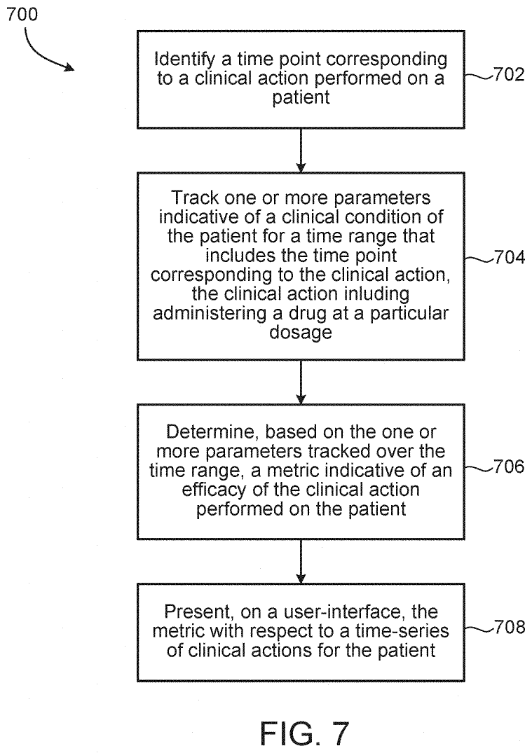

FIG. 7 is a flowchart of an example process 700 for calculating a medication efficacy score (MES). At least a portion of the process 700 can be implemented using one or more processors operation on the central server 102. Operations of the process 700 include identifying a time point corresponding to a clinical action performed on a patient (702) and tracking one or more parameters indicative of a clinical condition of the patient for a time range that includes the time point corresponding to the clinical action, wherein the clinical action includes administering a drug at a particular dosage (704). In some implementations, an end point of the time range is determined based on an expected time for the drug at the particular dosage to have a target effect. In some implementations, the one or more parameters tracked over the time range comprises at least one of: heart rate, blood pressure, venous pressure, oxygen saturation (SpO2), and pain level.

Operations of the process 700 also include determining, based on the one or more parameters tracked over the time range, a metric indicative of an efficacy of the clinical action performed on the patient (706). In some implementations, determining the metric includes applying a linear regression to the one or more parameters tracked over time. The process 700 further includes presenting, on a user-interface, the metric with respect to a time-series of clinical actions for the patient (708). In some implementations, the user-interface can include an identifier to indicate the method of administering the drug.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, one or more processors. The computer storage medium can be a machine-readable storage device, a machine-readable storage substrate, a random or serial access memory device, or a combination of one or more of them. Alternatively, or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus.

The term “processor” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them. One or more processors can be incorporated into or in communication with the servers, such as the central server 102, or other computing devices, such as the user computing device 110, the workstation 106, and/or the EMR database 104 described above.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user-interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

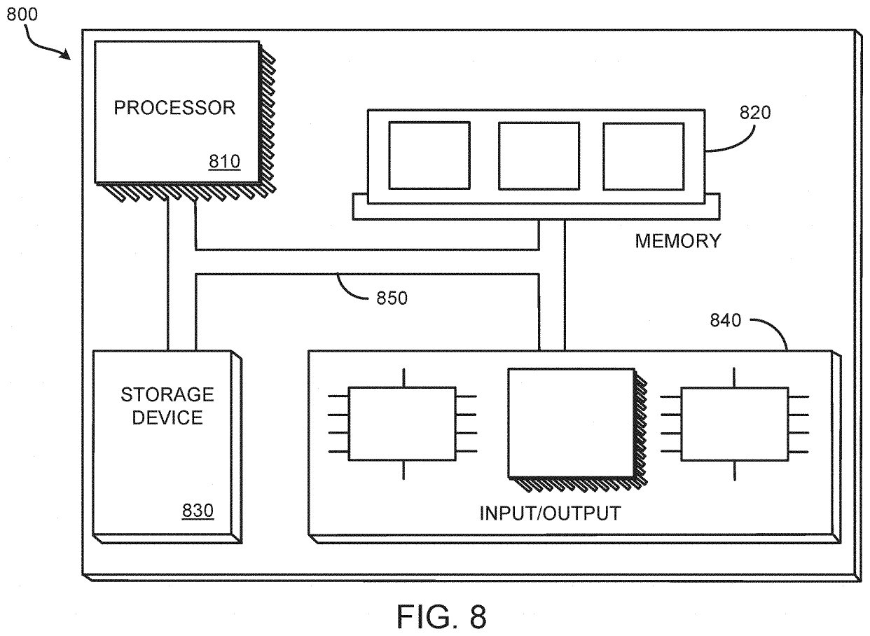

An example of one such type of computer is shown in FIG. 8, which shows a schematic diagram of a generic computer system 800. The system 800 can be used for the operations described in association with any of the computer-implemented methods described previously, according to one implementation. The system 800 includes a processor 810, a memory 820, a storage device 830, and an input/output device 840. Each of the components 810, 820, 830, and 840 are interconnected using a system bus 850. The processor 810 is capable of processing instructions for execution within the system 800. In one implementation, the processor 810 is a single-threaded processor. In another implementation, the processor 810 is a multi-threaded processor. The processor 810 is capable of processing instructions stored in the memory 820 or on the storage device 830 to display graphical information for a user-interface on the input/output device 840.

The memory 820 stores information within the system 800. In one implementation, the memory 820 is a computer-readable medium. In one implementation, the memory 820 is a volatile memory unit. In another implementation, the memory 820 is a non-volatile memory unit.

The storage device 830 is capable of providing mass storage for the system 800. In one implementation, the storage device 830 is a computer-readable medium. In various different implementations, the storage device 830 may be a floppy disk device, a hard disk device, an optical disk device, or a tape device.

The input/output device 840 provides input/output operations for the system 800. In one implementation, the input/output device 840 includes a keyboard and/or pointing device. In another implementation, the input/output device 840 includes a display unit for displaying graphical user-interfaces.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Other implementations are also within the scope of the following claims.

What is claimed is:

1. A method comprising:
obtaining, at one or more processing devices, a plurality of operating parameters of one or more respiratory support devices associated with a patient;
determining, by the one or more processing devices, based on the plurality of operating parameters, a first respiratory support type (RST) associated with the patient;
obtaining, by the one or more processing devices, a corresponding score range for the first respiratory support type;
determining, based at least on a subset of the plurality of operating parameters, a first respiratory support score in the corresponding score range, wherein the subset of the plurality of operating parameters is representative of intra-range variation within the corresponding score range; and
presenting, on a user-interface, the first respiratory support score as a part of a time-series of respiratory support scores for the patient.

2. The method of claim 1, wherein the plurality of operating parameters are obtained from one or more electronic medical records of the patient or medical devices associated with the patient.

3. The method of claim 1, wherein the plurality of operating parameters are obtained from the one or more respiratory support devices.

4. The method of claim 1, wherein the plurality of operating parameters comprises at least one numerical parameter selected from a group consisting of: fraction of inspired oxygen (FiO2), nasal cannula flow rate, aerosol mask flow rate, positive end-expiratory pressure (PEEP), positive inspiratory pressure (PIP), pressure support (PS), mandatory rate, set, mandatory and spontaneous tidal volume, high frequency oscillator ventilator (HFOV) frequency, HFOV amplitude, mean airway pressure, high frequency jet ventilator (HFJV) PEEP, HFJV PIP, HFJV rate, extracorporeal membrane oxygen (ECMO) time, ECMO flow, and amount of inhaled nitric oxide.

5. The method of claim 1, wherein the plurality of operating parameters comprises at least one parameter selected from a group consisting of: a bilevel positive airway pressure (BiPAP) identifier; a continuous positive airway pressure (CPAP) identifier; a ventilator mode (VM) identifier, an extracorporeal membrane oxygen (ECMO) type identifier; an identifier of the patency of an artificial airway; an identifier of the use of an artificial airway; and a respiratory support device identifier.

6. The method of claim 1, further comprising:
determining that the first respiratory support score satisfies a threshold condition; and
responsive to determining that the first respiratory support score satisfies a threshold condition, generating an alert indicative of the first respiratory support score.

7. The method of claim 1, wherein the corresponding score range for the first respiratory support type depends on an age of the patient.

8. The method of claim 1, wherein the corresponding score range for the first respiratory support type depends on a clinical condition of the patient.

9. The method of claim 1, wherein determining the RST comprises:
identifying that at least two of the plurality of operating parameters provide conflicting information usable in determining the first RST;
identifying at least a second RST corresponding to a time point within a threshold distance in the time series; and
determining the first RST to be same as the second RST.

10. The method of claim 1, wherein the first respiratory support type is determined as one of a group consisting of: room air, nasal cannula, aerosol mask, high-flow nasal cannula (HFNC), continuous positive airway pressure (CPAP), bilevel positive airway pressure (BiPAP), pressure support (PSV), pressure control ventilation (PCV), pressure support (PS), volume control ventilation (VCV) high frequency oscillator ventilator (HFOV), high frequency jet ventilator (HFJV), and venoarterial or venovenous extracorporeal membrane oxygen (ECMO).

11. The method of claim 1, wherein the user-interface identifies intubation and extubation time points with respect to the time-series of the respiratory support scores.

12. The method of claim 1, wherein the user-interface identifies a location of the patient with respect to the time-series of the respiratory support scores.

13. The method of claim 1, further comprising:
presenting, on the user-interface, patient clinical data, wherein the patient clinical data is overlaid on the time-series of respiratory support scores such that the patient clinical data shares the same time-series as the respiratory support scores.

14. The method of claim 1, further comprising:
identifying a time point corresponding to a clinical action performed on the patient;
tracking one or more parameters indicative of a clinical condition of the patient for a time range that includes the time point corresponding to the clinical action, wherein the clinical condition is expected to be affected by the clinical action; and
representing, on the user-interface, changes to the one or more parameters over the time range with respect to the time-series of the respiratory support scores.

15. The method of claim 14, further comprising:
determining, based on the one or more parameters tracked over the time range, a metric indicative of an efficacy of the clinical action performed on the patient; and
presenting the metric on the user-interface with respect to the time-series of the respiratory support scores.

16. The method of claim 15, wherein the clinical action comprises administering a drug at a particular dosage.

17. The method of claim 16, wherein an end point of the time range is determined based on an expected time for the drug at the particular dosage to have a target effect.

18. The method of claim 14, wherein the one or more parameters tracked over the time range comprises at least one of: heart rate, blood pressure, venous pressure, oxygen saturation (SpO₂), and pain level.

19. A method comprising:
identifying a time point corresponding to a clinical action performed on a patient;
tracking one or more parameters indicative of a clinical condition of the patient for a time range that includes the time point corresponding to the clinical action, wherein the clinical action comprises administering a drug at a particular dosage;
determining, based on the one or more parameters tracked over the time range, a metric indicative of an efficacy of the clinical action performed on the patient; and
presenting, on a user-interface, the metric with respect to a time-series of clinical actions for the patient.

20. The method of claim 19, wherein an end point of the time range is determined based on an expected time for the drug at the particular dosage to have a target effect.

21. The method of claim 19, wherein the one or more parameters tracked over the time range comprises at least one of: heart rate, blood pressure, oxygen saturation (SpO₂), and pain level.

22. The method of claim 19, wherein determining the metric comprises applying a linear regression to the one or more parameters tracked over time.

23. A system comprising:
memory; and
one or more processing devices configured to:
obtain a plurality of operating parameters of one or more respiratory support devices associated with a patient;
determine based on the plurality of operating parameters, a first respiratory support type (RST) associated with the patient;
obtain a corresponding score range for the first respiratory support type;
determine, based at least on a subset of the plurality of operating parameters, a first respiratory support score in the corresponding score range, wherein the subset of the plurality of operating parameters is representative of intra-range variation within the corresponding score range; and
present, on a user-interface, the first respiratory support score as a part of a time-series of respiratory support scores for the patient.

24. The system of claim 23, wherein the plurality of operating parameters are obtained from one or more electronic medical records of the patient or medical devices associated with the patient.

25. The system of claim 23, wherein the plurality of operating parameters are obtained from the one or more respiratory support devices.

26. The system of claim 23, wherein the plurality of operating parameters comprises at least one numerical parameter selected from a group consisting of: fraction of inspired oxygen (FiO2), nasal cannula flow rate, aerosol mask flow rate, positive end-expiratory pressure (PEEP), positive inspiratory pressure (PIP), pressure support (PS), mandatory rate, set, mandatory and spontaneous tidal volume, high frequency oscillator ventilator (HFOV) frequency, HFOV amplitude, mean airway pressure, high frequency jet ventilator (HFJV) PEEP, HFJV PIP, HFJV rate, extracorporeal membrane oxygen (ECMO) time, ECMO flow, and amount of inhaled nitric oxide.

27. The system of claim 23, wherein the plurality of operating parameters comprises at least one parameter selected from a group consisting of: a bilevel positive airway pressure (BiPAP) identifier; a continuous positive airway pressure (CPAP) identifier; a ventilator mode (VM) identifier, an extracorporeal membrane oxygen (ECMO) type identifier; an identifier of the patency of an artificial airway; an identifier of the use of an artificial airway; and a respiratory support device identifier.

28. The system of claim 23, wherein the one or more processing devices are configured to:
determine that the first respiratory support score satisfies a threshold condition; and
responsive to determining that the first respiratory support score satisfies a threshold condition, generate an alert indicative of the first respiratory support score.

29. The system of claim 23, wherein the corresponding score range for the first respiratory support type depends on an age of the patient.

30. The system of claim 23, wherein the corresponding score range for the first respiratory support type depends on a clinical condition of the patient.

31. The system of claim 23, wherein determining the RST comprises:
identifying that at least two of the plurality of operating parameters provide conflicting information usable in determining the first RST;
identifying at least a second RST corresponding to a time point within a threshold distance in the time series; and
determining the first RST to be same as the second RST.

32. The system of claim 23, wherein the first respiratory support type is determined as one of a group consisting of: room air, nasal cannula, aerosol mask, high-flow nasal cannula (HFNC), continuous positive airway pressure (CPAP), bilevel positive airway pressure (BiPAP), pressure support (PSV), pressure control ventilation (PCV), pressure support (PS), volume control ventilation (VCV) high frequency oscillator ventilator (HFOV), high frequency jet ventilator (HFJV), and venoarterial or venovenous extracorporeal membrane oxygen (ECMO).

33. The system of claim 23, wherein the user-interface identifies intubation and extubation time points with respect to the time-series of the respiratory support scores.

34. The system of claim 23, wherein the user-interface identifies a location of the patient with respect to the time-series of the respiratory support scores.

35. The system of claim 23, wherein the one or more processing devices are configured to:
present, on the user-interface, patient clinical data, wherein the patient clinical data is overlaid on the time-series of respiratory support scores such that the patient clinical data shares the same time-series as the respiratory support scores.

36. The system of claim 23, wherein the one or more processing devices are configured to:
identify a time point corresponding to a clinical action performed on the patient;
track one or more parameters indicative of a clinical condition of the patient for a time range that includes the time point corresponding to the clinical action, wherein the clinical condition is expected to be affected by the clinical action; and
represent, on the user-interface, changes to the one or more parameters over the time range with respect to the time-series of the respiratory support scores.

37. The system of claim 36, wherein the one or more processing devices are configured to:
determine, based on the one or more parameters tracked over the time range, a metric indicative of an efficacy of the clinical action performed on the patient; and
present the metric on the user-interface with respect to the time-series of the respiratory support scores.

38. The system of claim 37, wherein the clinical action comprises administering a drug at a particular dosage.

39. The system of claim 38, wherein an end point of the time range is determined based on an expected time for the drug at the particular dosage to have a target effect.

40. The system of claim 36, wherein the one or more parameters tracked over the time range comprises at least one of: heart rate, blood pressure, venous pressure, oxygen saturation (SpO2), and pain level.

41. A system comprising:
memory; and
one or more processing devices configured to:
identify a time point corresponding to a clinical action performed on a patient;
track one or more parameters indicative of a clinical condition of the patient for a time range that includes the time point corresponding to the clinical action, wherein the clinical action comprises administering a drug at a particular dosage;
determine, based on the one or more parameters tracked over the time range, a metric indicative of an efficacy of the clinical action performed on the patient; and
present, on a user-interface, the metric with respect to a time-series of clinical actions for the patient.

42. The system of claim 41, wherein an end point of the time range is determined based on an expected time for the drug at the particular dosage to have a target effect.

43. The system of claim 41, wherein the one or more parameters tracked over the time range comprises at least one of: heart rate, blood pressure, oxygen saturation (SpO2), and pain level.

44. The system of claim 41, wherein determining the metric comprises applying a linear regression to the one or more parameters tracked over time.

45. One or more machine-readable storage devices having encoded thereon computer readable instructions for causing one or more processing devices to perform operations comprising:
obtaining a plurality of operating parameters of one or more respiratory support devices associated with a patient;
determining based on the plurality of operating parameters, a first respiratory support type (RST) associated with the patient;
obtaining a corresponding score range for the first respiratory support type;
determining, based at least on a subset of the plurality of operating parameters, a first respiratory support score in the corresponding score range, wherein the subset of the plurality of operating parameters is representative of intra-range variation within the corresponding score range; and
presenting, on a user-interface, the first respiratory support score as a part of a time-series of respiratory support scores for the patient.

46. The one or more machine-readable storage devices of claim 45, wherein the plurality of operating parameters are obtained from one or more electronic medical records of the patient or medical devices associated with the patient.

47. The one or more machine-readable storage devices of claim 45, wherein the plurality of operating parameters are obtained from the one or more respiratory support devices.

48. The one or more machine-readable storage devices of claim 45, wherein the plurality of operating parameters comprises at least one numerical parameter selected from a group consisting of: fraction of inspired oxygen (FiO2), nasal cannula flow rate, aerosol mask flow rate, positive end-expiratory pressure (PEEP), positive inspiratory pressure (PIP), pressure support (PS), mandatory rate, set, mandatory and spontaneous tidal volume, high frequency oscillator ventilator (HFOV) frequency, HFOV amplitude, mean airway pressure, high frequency jet ventilator (HFJV) PEEP, HFJV PIP, HFJV rate, extracorporeal membrane oxygen (ECMO) time, ECMO flow, and amount of inhaled nitric oxide.

49. The one or more machine-readable storage devices of claim 45, wherein the plurality of operating parameters comprises at least one parameter selected from a group consisting of: a bilevel positive airway pressure (BiPAP) identifier; a continuous positive airway pressure (CPAP) identifier; a ventilator mode (VM) identifier, an extracorporeal membrane oxygen (ECMO) type identifier; an identifier of the patency of an artificial airway; an identifier of the use of an artificial airway; and a respiratory support device identifier.

50. The one or more machine-readable storage devices of claim 45 having encoded thereon additional computer readable instructions for causing the one or more processing devices to perform operations comprising:
determining that the first respiratory support score satisfies a threshold condition; and
responsive to determining that the first respiratory support score satisfies a threshold condition, generating an alert indicative of the first respiratory support score.

51. The one or more machine-readable storage devices of claim 45, wherein the corresponding score range for the first respiratory support type depends on an age of the patient.

52. The one or more machine-readable storage devices of claim 45, wherein the corresponding score range for the first respiratory support type depends on a clinical condition of the patient.

53. The one or more machine-readable storage devices of claim 45, wherein determining the RST comprises:
identifying that at least two of the plurality of operating parameters provide conflicting information usable in determining the first RST;
identifying at least a second RST corresponding to a time point within a threshold distance in the time series; and
determining the first RST to be same as the second RST.

54. The one or more machine-readable storage devices of claim 45, wherein the first respiratory support type is determined as one of a group consisting of: room air, nasal cannula, aerosol mask, high-flow nasal cannula (HFNC), continuous positive airway pressure (CPAP), bilevel positive airway pressure (BiPAP), pressure support (PSV), pressure control ventilation (PCV), pressure support (PS), volume control ventilation (VCV) high frequency oscillator ventilator (HFOV), high frequency jet ventilator (HFJV), and venoarterial or venovenous extracorporeal membrane oxygen (ECMO).

55. The one or more machine-readable storage devices of claim 45, wherein the user-interface identifies intubation and extubation time points with respect to the time-series of the respiratory support scores.

56. The one or more machine-readable storage devices of claim 45, wherein the user-interface identifies a location of the patient with respect to the time-series of the respiratory support scores.

57. The one or more machine-readable storage devices of claim 45 having encoded thereon additional computer readable instructions for causing the one or more processing devices to perform operations comprising:
presenting, on the user-interface, patient clinical data, wherein the patient clinical data is overlaid on the time-series of respiratory support scores such that the patient clinical data shares the same time-series as the respiratory support scores.

58. The one or more machine-readable storage devices of claim 45 having encoded thereon additional computer readable instructions for causing the one or more processing devices to perform operations comprising:
identifying a time point corresponding to a clinical action performed on the patient;
tracking one or more parameters indicative of a clinical condition of the patient for a time range that includes the time point corresponding to the clinical action, wherein the clinical condition is expected to be affected by the clinical action; and
representing, on the user-interface, changes to the one or more parameters over the time range with respect to the time-series of the respiratory support scores.

59. The one or more machine-readable storage devices of claim 58 having encoded thereon additional computer readable instructions for causing the one or more processing devices to perform operations comprising:
determining, based on the one or more parameters tracked over the time range, a metric indicative of an efficacy of the clinical action performed on the patient; and
presenting the metric on the user-interface with respect to the time-series of the respiratory support scores.

60. The one or more machine-readable storage devices of claim 59, wherein the clinical action comprises administering a drug at a particular dosage.

61. The one or more machine-readable storage devices of claim 60, wherein an end point of the time range is determined based on an expected time for the drug at the particular dosage to have a target effect.

62. The one or more machine-readable storage devices of claim 58, wherein the one or more parameters tracked over the time range comprises at least one of: heart rate, blood pressure, venous pressure, oxygen saturation (SpO₂), and pain level.

63. One or more machine-readable storage devices having encoded thereon computer readable instructions for causing one or more processing devices to perform operations comprising:
identifying a time point corresponding to a clinical action performed on a patient;
tracking one or more parameters indicative of a clinical condition of the patient for a time range that includes the time point corresponding to the clinical action, wherein the clinical action comprises administering a drug at a particular dosage;
determining, based on the one or more parameters tracked over the time range, a metric indicative of an efficacy of the clinical action performed on the patient; and
presenting, on a user-interface, the metric with respect to a time-series of clinical actions for the patient.

64. The one or more machine-readable storage devices of claim 63 wherein an end point of the time range is determined based on an expected time for the drug at the particular dosage to have a target effect.

65. The one or more machine-readable storage devices of claim 63, wherein the one or more parameters tracked over the time range comprises at least one of: heart rate, blood pressure, oxygen saturation (SpO₂), and pain level.

66. The one or more machine-readable storage devices of claim 63 wherein determining the metric comprises applying a linear regression to the one or more parameters tracked over time.
